(19) United States
(12) Patent Application Publication
(10) Pub. No.: US 2006/0167414 A1
(43) Pub. Date: Jul. 27, 2006

(54) Syringe pump with barrel size sensor arm

(76) Inventors: Adrian Paul Scott, London (GB); Steven Christopher Hart, Kimpton (GB)

Correspondence Address: LOUIS WOO;LAW OFFICE OF LOUIS WOO, 717 NORTH FAYETTE STREET, ALEXANDRIA, VA, 22314, US

(21) Appl. No.: 10/560,965

(22) PCT Filed: Jul. 2, 2004
(86) PCT No.: PCT/GB04/02878

§ 371 Date: Dec. 16, 2005

(30) Foreign Application Priority Data
Jul. 9, 2003 (GB) 03161601

(51) Int. Cl. A61M 1/00

(52) U.S. Cl. 604/152

(57) ABSTRACT

A syringe pump has a pusher (86) that pushes in the plunger (81) of a syringe (8) to dispense medication. A pivoted barrel size sensor arm (201) engages the syringe barrel (82) at one end (203) and carries three flags (207, 208 and 209) at the other end (205), which move across three sensors (217, 218 and 219) such that each sensor can change from a first state to a second state and back to a first state as the arm moves, to provide nine different size outputs.

This invention relates to syringe pumps of the kind including a syringe barrel size sensor mechanism including a movable member mounted for contact with the syringe barrel, a plurality of sensors and a plurality of flags, the movable member being arranged to effect relative movement between the flags and the sensors.

Syringe pumps are used to administer liquid medication to a patient at a precisely controlled rate. A syringe is filled with medication and is connected to a catheter. The syringe is then loaded into the pump and its plunger is gripped by a pusher mechanism, which is moved forwardly to push in the plunger and dispense the medication. Usually, the pusher is moved forwardly by means of a leadscrew rotated by a motor.

It is known in such pumps to have some arrangement for measuring the diameter of the syringe barrel so that the size of the syringe can be determined. The pump displays an indication of the syringe size for the user to confirm that this is correct. This information is then used in controlling the rate at which medication is dispensed. An example of a syringe barrel size measuring arrangement is described in GB2350062 where a strip coupled with an arm that bears on the syringe barrel moves along a CCD array of over a hundred elements. The strip has apertures of different lengths and, by measuring the position and length of the apertures, it is possible to obtain a very accurate indication of the diameter of the syringe barrel. Although this arrangement is very accurate it is relatively expensive and requires calibration, which does not make it suitable for low cost pumps. Alternative, low cost, arrangements involve a flag moving along a row of typically three sensors so that an increasing number of 1, 2 or 3 sensors are obscured as the flag moves. This arrangement gives an approximate indication of size but it does not enable the pump to distinguish between many different syringes.

Syringe pumps also commonly have some arrangement for detecting that the head of the plunger has been correctly retained by the pusher. This is important because, if the plunger is not retained, it is possible that it could move forwardly in an uncontrolled manner along the barrel and allow medication to siphon out of the syringe. The usual arrangement for detecting the presence of the plunger head is some form of electrical switch or pressure sensor in the pusher, such as described in GB2368288. Although this arrangement can function satisfactorily, it involves electrical connection being made to the movable pusher, which can cause problems, especially because the pusher may be exposed to liquid.

It is an object of the present invention to provide an alternative syringe pump.

According to the present invention there is provided a syringe pump of the above-specified kind, characterised in that each sensor is responsive to a flag to provide a first or second output according to the position of the flag relative to the sensor, and that the flags and sensors are arranged such that the output of at least one sensor changes from the first to the second state and then back to the first state for relative movement between the flags and sensors in one direction.

Preferably all the sensors change from the first to the second state and then back to the first state for relative movement between the flags and the sensors. The flags and sensors may be arranged such that the movable member is movable between a position in which a sensor is exposed on one side of a flag, through a second position where the flag is aligned with the sensor to a third position where the sensor is exposed on an opposite side of the flag. The movabel member may include an arm pivotally mounted at one end and having its other end arranged to contact the syringe barrel. The movable member may have an overcentre action. The flags are preferably movable with the movable member, the sensors being fixed with the pump housing. The sensors are preferably optical sensors. The barrel size sensor mechanism preferably includes three sensors and three flags.

A syringe pump according to the present invention will now be described, by way of example, with reference to the accompanying drawings, in which:

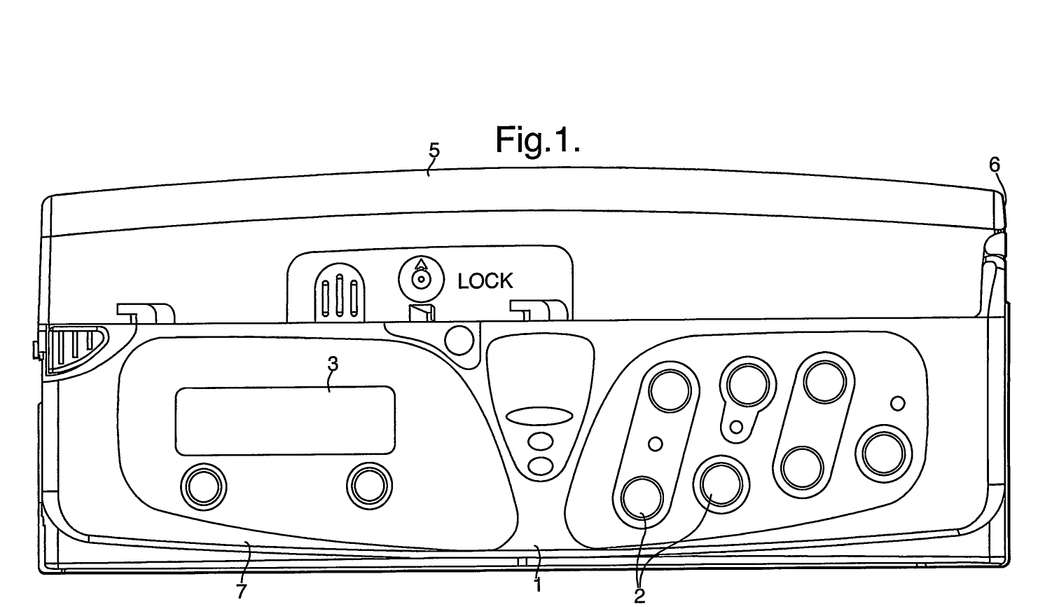

FIG. 1 is an elevation view of the front of the pump;

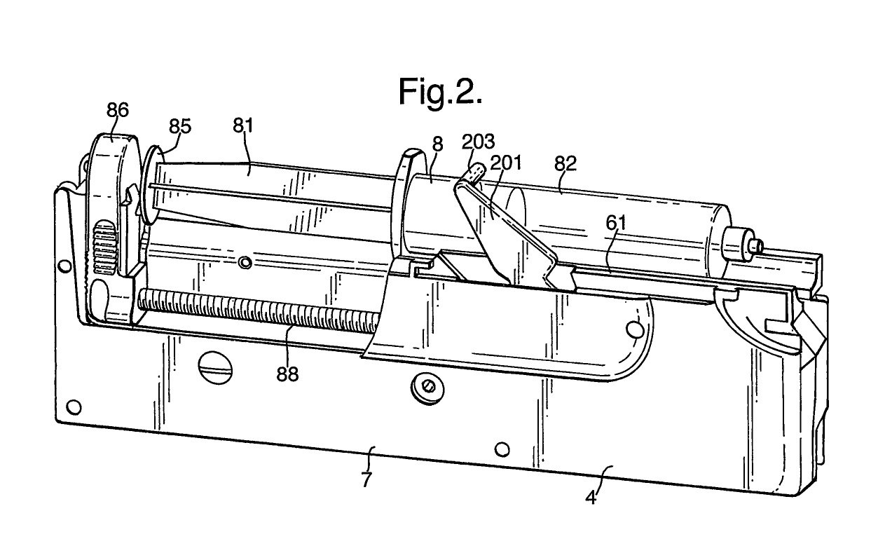

FIG. 2 is perspective view of the rear of the pump with a part of its casing removed;

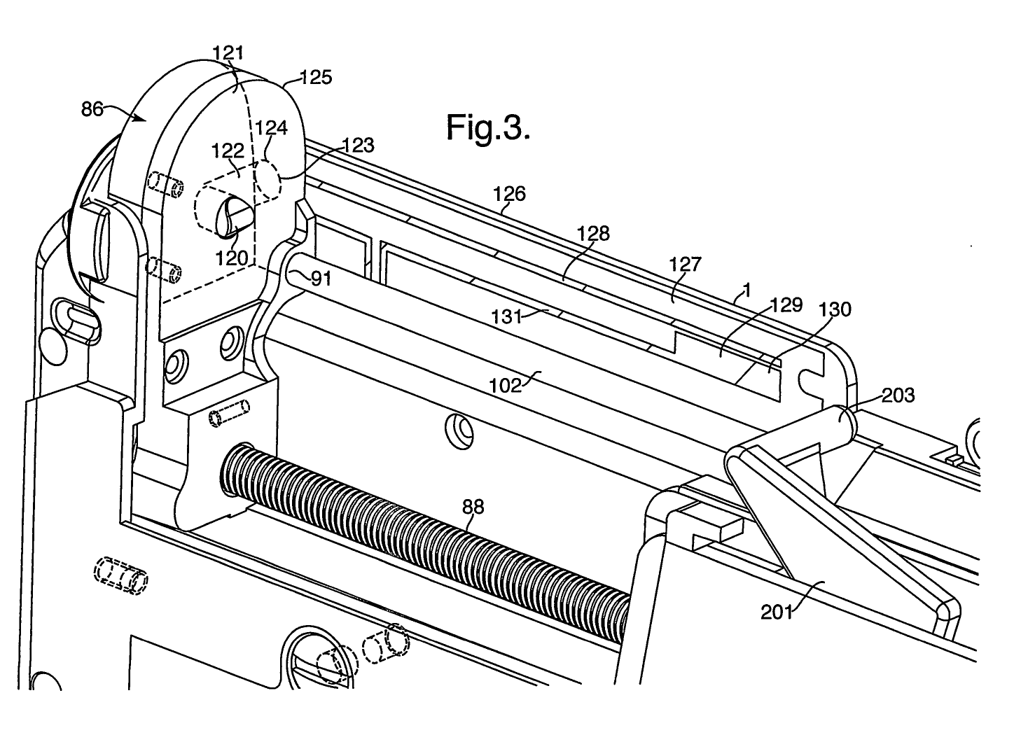

FIG. 3 is a perspective view from the rear showing parts of the pump pusher mechanism;

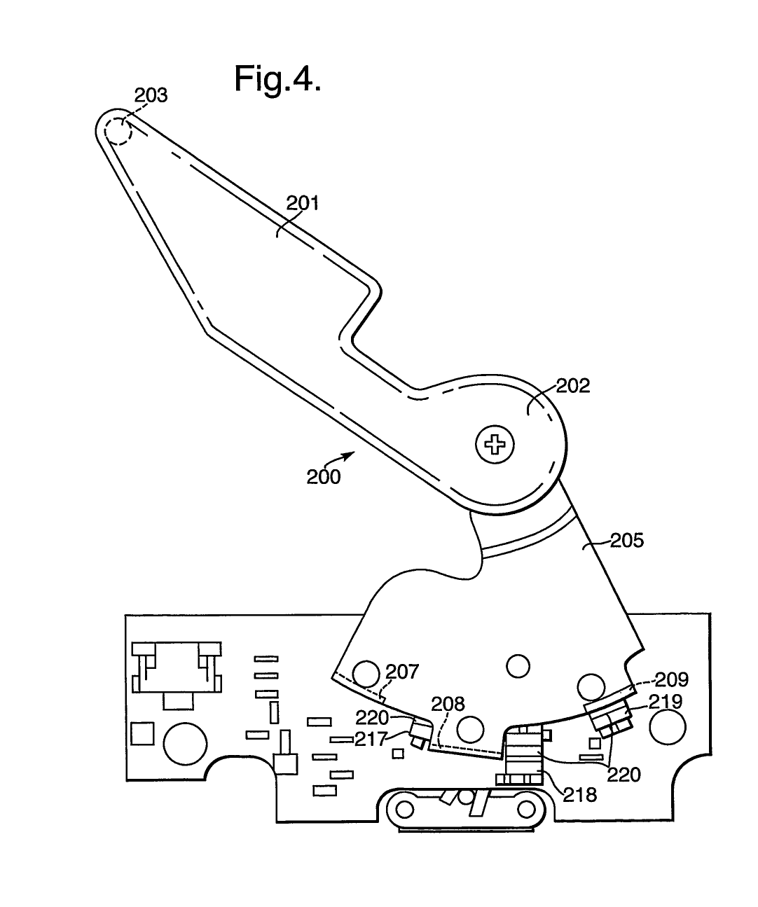

FIG. 4 is an elevation view of the rear of barrel size sensor mechanism; and

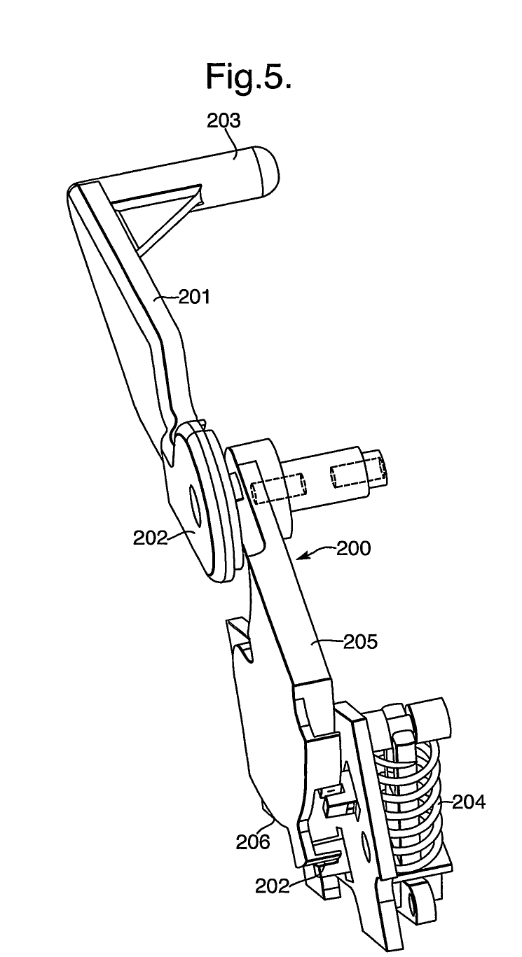

FIG. 5 is a perspective view of the barrel size sensor mechanism.

With reference first to FIGS. 1 and 2, the pump has a generally rectangular shape with a substantially flat front face 1 supporting various controls 2 and a display 3. The rear surface 4 is also substantially flat and may have formations (not shown) for supporting the pump in a horizontal attitude.

The upper surface 61 of the casing 7 supports a conventional syringe 8 beneath a cover 5 hinged at its right-hand end 6 on the casing. The syringe 8 is shown with its plunger 81 in an extended position relative to the barrel 82, that is, with the syringe full of medication. The head 85 of the plunger 81 is gripped by a plunger pusher mechanism indicated generally by the numeral 86 and shown in more detail in FIG. 3. The parts of the pusher mechanism 86 that grip the plunger head 85 have been omitted from FIG. 3 for clarity. The pusher mechanism 86 is moved by a lead screw 88 extending lengthwise of the casing 7 and rotated axially by means of a motor (not shown) in the usual manner. The pusher 86 has an aperture 91 through which a guide rod 102 extends parallel to the lead screw 88. The guide rod 102 prevents rotation of the pusher 86 and ensures that rotation of the lead screw 88 is translated entirely into axial movement of the plunger 81, thereby ensuring accurate medication delivery.

A spring-loaded peg 120 projects from the forward surface 121 of the pusher mechanism 86 so that this engages and is pushed rearwardly by contact with the rear surface of the head 85 of the plunger 81 when this is correctly captured by the pusher mechanism. The peg 120 is coupled within the mechanism 86 to a laterally-extending rod 122 extending at right angles to the direction of travel, towards the front face 1 of the pump. The outer end 123 of the rod 122 is rounded and aligned with an aperture 124 in the side 125 of the pusher mechanism 86. With the peg 120 in its natural, extended position shown in FIG. 3, that is, with no plunger head retained, the end 123 of the rod 122 is level with the side 125 of the pusher mechanism 86. When the peg 120 is pushed in by engagement with the plunger 81, the rod 122 is pushed laterally outwardly so that its end 123 projects a small distance through the aperture 124.

The side 125 of the pusher mechanism 86 moves along a panel 126 extending along the inside of the front face 1 of the pump. The inner surface 127 of the panel 126, that is, the surface adjacent the pusher mechanism 86, supports two membrane switches 128 and 129. The first membrane switch 128 takes the form of an elongate narrow strip of constant width extending horizontally parallel to the direction of travel of the pusher mechanism. The first switch 128 is positioned vertically so that it aligns with the upper part of the aperture 124 in the pusher mechanism 86. The second switch 129 has an operative contact region 130 about three times the width of the first switch 128 and a length of about 20 mm. The contact region 130 is located below the first switch 128 and towards its right-hand, forward end. The contact region 130 connects with a thinner track 131 extending rearwardly and spaced below the first switch 128. The disposition of the first switch 128 is such that it will be contacted at any point along its length by the end 123 of the rod 122 when this is pushed out by engagement with the plunger 81. The force with which the rod 122 is pushed out is sufficient to ensure that the switch 128 is turned on. The contacting surfaces of the rod 122 and switch 128 have a low friction so there is little resistance to forward movement of the plunger mechanism 86. As the pusher mechanism 86 moves forwardly, the end 123 of the rod 122 slides along the switch 128 making continuous contact with it and keeping the switch actuated for as long as the plunger 81 is correctly engaged with the pusher mechanism. The end 123 of the rod 122 is spaced above the track 131 of the second switch 129 for all rear positions of the pusher mechanism 86 so that the second switch remains off. When the pusher mechanism 86 approaches close to the limit of its forward travel, the end 123 of the rod 122 contacts the rear end of the enlarged contact region 130 of the second switch 129, thereby turning it on and providing an output indicating that the syringe 8 is nearly empty. The second switch 129 remains on as the pusher mechanism 86 moves forwardly along the length of the contact region 130.

This arrangement enables an output indication to be provided indicative of both plunger capture and a near empty syringe without the need for any electrical connection to the moving components. The membrane switches 128 and 129 are completely enclosed electrically so are not damaged by contact with fluid. It will be appreciated that the pusher could instead be arranged such that the rod 122 is displaced outwardly when the pusher is disengaged from the plunger, in which case, when the switch 128 is contacted by the rod, the output of the switch would be indicative of a fault.

With reference now also to FIGS. 4 and 5, the pump has a syringe size sensor assembly 200 including an arm 201 extending upwardly at an angle and hinged towards its lower end 202 about a horizontal axis. The arm 201 extends up the rear side of the syringe 8 and has a transverse finger 203 at its upper end extending forwardly above the barrel 82 at least across half the diameter of the barrel. An overcentre spring arrangement 204 urges the arm 201 anticlockwise, as viewed in FIGS. 2, 4 and 5, so that the finger 203 is urged down on the syringe barrel 82. The overcentre action enables the arm 201 to be retained in an open position by rotating it clockwise past a vertical position. The lower end 202 of the arm 201 is coupled with a sector plate 205, on the opposite side of the axis of rotation, which has a curved lower surface 206 supporting three flags 207, 208 and 209 spaced from one another along the lower surface. The flags 207 to 209 are provided by forwardly-projecting curved plates formed from the sector plate 205, which is of an opaque material. The two outer flags 207 and 209 are at the same radial distance from the upper, pivoted end of the sector plate 205 whereas the middle flag 208 is spaced radially outwardly by a short distance. The three flags are arranged to cooperate with three sensors 217, 218 and 219 respectively, which are optical transmission sensors each having a slot 220 between an emitter and receiver. The flags 207 to 209 are located to pass through the slot 220 of respective sensors 217 to 219 as the arm 201 rotates, thereby interrupting the optical path and changing the output of the sensor. The length of the flags 207 to 209 and their positions are such that the arm 201 is initially in a first position where at least one sensor is exposed on one side of a flag. Then the arm 201 moves through a second position where the flag is aligned with and obscures the sensor, thereby changing its output. Continued movement of the arm 201 moves it to a third position where the sensor is exposed on an opposite side of the flag. This thereby causes at least one of the sensors 217 to 219 to change from an initial state, to a second state and then back to an initial state as the arm moves in one direction.

In particular, if the sensors 217, 218 and 219 are designated A, B and C respectively, as the arm 201 rotates clockwise when viewed from the rear of the pump, from the smallest syringe diameter to the largest, their outputs will be as follows:

| A | B | C |
|---|---|---|
| 1 | 0 | 0 |
| 1 | 1 | 0 |
| 1 | 1 | 1 |
| 0 | 1 | 1 |
| 0 | 0 | 1 |
| 0 | 0 | 0 |

It can be seen that both sensors B and C change from “0” to “1” and then back to “0” as the arm moves in one direction. This arrangement enables discrimination between six different angles of the arm 201. It will be appreciated, however, that it would be possible with three sensors and three flags to have up to nine different sensor outputs, that is, 3². In general, where n flags and sensors are used it would be possible to provide a maximum of n² different outputs. The control unit of the pump readily converts the sensor outputs into an angle measurement and hence into a measurement of the diameter of the syringe barrel 82.

This arrangement enables the pump to distinguish between a greater number of diameters of syringe than would be possible using the same number of sensors in a conventional fashion. The arrangement, however, is low cost and does not require calibration.

The sensors need not be optical sensors but could, for example, be of a magnetic or any other suitable form.

1. A syringe pump including a syringe barrel size sensor mechanism including a movable member mounted for contact with the syringe barrel, a plurality of sensors and a plurality of flags, the movable member being arranged to effect relative movement between the flags and the sensors, characterized in that each sensor is responsive to a flag to provide a first or second output according to the position of the flag relative to the sensor, and that the flags and sensors are arranged such that the output of at least one sensor changes from the first to the second state and then back to the first state for relative movement between the flags and sensors in one direction.

2. A syringe pump according to claim 1, characterized in that all of the sensors change from the first to the second state and then back to the first state for relative movement between the flags and the sensors.

3. A syringe pump according to claim 1, characterized in that the flags and sensors are arranged such that the movable member is movable between a position in which a sensor is exposed on one side of a flag, through a second position where the flag is aligned with the sensor to a third position where the sensor is exposed on an opposite side of the flag.

4. A syringe pump according to claim 1, characterized in that the movable member includes an arm pivotally mounted at one end and having its other end arranged to contact the syringe barrel.

5. A syringe pump according to claim 1, characterized in that the movable member has an overcentre action.

6. A syringe pump according to claim 1, characterized in that the flags are movable with the movable member and that the sensors are fixed with the pump housing.

7. A syringe pump according to claim 1, characterized in that the sensors are optical sensors.

8. A syringe pump according to claim 1, characterized in that the barrel size sensor mechanism includes three sensors and three flags.
